SEQUENCE LISTING (12 sequences)

SEQ ID NO: 1 - DNA, 90 bp, Artificial Sequence
  Hairpin Adaptor
actctttccctacacgacgctcttccgatcttcgcattccgatctcatctagagccaccagcggcatagtagctagaagcgtaaggctag

SEQ ID NO: 2 - DNA, 90 bp, Artificial Sequence
  Hairpin Adaptor
actctttccctacacgacgctcttccgatcttcgcattccgatcccatctagagccaccagcggcatagtagctagaagcgtaaggctag

SEQ ID NO: 3 - DNA, 43 bp, Artificial Sequence
  The 5′ end first nucleotide A does not include a phosphate group
agctagaagcgtaaggctagcatcgatcttcgcattccgatct

SEQ ID NO: 4 - DNA, 92 bp, Artificial Sequence
  The 5′ end first nucleotide G includes a phosphate group
gatcggaatgcgaagatcgatgacttacggcgacgagatctacttctcgcagcaacccatccctttcagtcctagccttacgcttctagctt

SEQ ID NO: 5 - DNA, 43 bp, Artificial Sequence
  The 5′ end first nucleotide A does not include a phosphate group
agctagaagcgtaaggctagcatcgatcttcgcattccgatcc

SEQ ID NO: 6 - DNA, 92 bp, Artificial Sequence
  The 5′ end first nucleotide G includes a phosphate group
gatcggaatgcgaagatcgatgacttacggcgacgagatctacttctcgcagcaacccatccctttcagtcctagccttacgcttctagctc

SEQ ID NO: 7 - DNA, 43 bp, Artificial Sequence; identical to SEQ ID NO: 3
  The 5′ end first nucleotide A includes a phosphate group

SEQ ID NO: 8 - DNA, 92 bp, Artificial Sequence; identical to SEQ ID NO: 4
  The 5′ end first nucleotide G includes a phosphate group

SEQ ID NO: 9 - DNA, 43 bp, Artificial Sequence; identical to SEQ ID NO: 5
  The 5′ end first nucleotide A includes a phosphate group

SEQ ID NO: 10 - DNA, 92 bp, Artificial Sequence; identical to SEQ ID NO: 6
  The 5′ end first nucleotide G includes a phosphate group

SEQ ID NO: 11 - DNA, 22 bp, Artificial Sequence
  Oligo Primer
gagcgtcgtgtagggaaagagt

SEQ ID NO: 12 - DNA, 26 bp, Artificial Sequence
  Oligo Primer
tgggttgctgcgagaagtagatctcg

(19) United States
(12) Patent Application Publication
(10) Pub. No.: US 2018/0100180 A1
(43) Pub. Date: Apr. 12, 2018

(54) METHODS OF SINGLE DNA/RNA MOLECULE COUNTING

(71) Applicant: Silgentech Inc., Sunnyvale, CA (US)

(72) Inventor: Meihong LIN, Redwood City, CA (US)

(21) Appl. No.: 15/358,076

(22) Filed: Nov. 21, 2016

Related U.S. Application Data
(60) Provisional application No. 62/414,628, filed on Oct. 28, 2016.
(60) Provisional application No. 62/275,177, filed on Jan. 5, 2016.

(51) Int. Cl. C12Q 1/68

(52) U.S. Cl. CPC C12Q 1/682; C12Q 1/6855

(57) ABSTRACT

A method for counting single- or double-stranded polynucleotide molecules is provided. A plurality of double-stranded polynucleotide fragments is obtained from original single- or double-stranded polynucleotide molecules. The ends of the polynucleotide fragments can be modified to have overhangs suitable for ligation. Two single-stranded adaptors or one double-stranded polynucleotide linker are ligated to both ends of each of the double-stranded polynucleotide fragments to form single- or double-stranded circular polynucleotide molecules, which can be linearly amplified to form single-stranded nanoballs. The circular polynucleotide molecules before the amplification, or the single-stranded nanoballs obtained in the amplification, can be identified and quantified on a gene detection platform, thereby obtaining a counting of the original single- or double-stranded polynucleotide molecules.

### CROSS REFERENCE TO RELATED APPLICATIONS

This application claims priority to U.S. Provisional Application No. 62/275,177, filed Jan. 5, 2016, and U.S. Provisional Application No. 62/414,628, filed Oct. 28, 2016, the disclosure of each of which is incorporated by reference herein in its entirety.

### FIELD

The present invention relates to DNA and RNA identification and quantification.

### BACKGROUND

Single molecule detection or digital counting technologies such as digital PCR and single molecule sequencing have great advantages over conventional methods for genetic mutation detection and quantification since the former can identify and quantitate genes at a single molecule resolution.

However, even these new technologies still suffer some deficiencies. For example, in single molecule sequencing, multiple linear copies of the same molecule need to be sequenced for sequencing error correction, or each gene fragment is amplified into numerous separate, unconnected copies which usually introduce errors and biased sequence representation before sequencing. Moreover, the operation procedures for library construction are complex, time-consuming and tedious, requiring purification which results in sample loss, and the compositions of the libraries are highly redundant.

### SUMMARY

The present invention provides for methods of generating DNA nanorings/nanoballs, which may be used for counting DNA or RNA molecules at a single molecule resolution on different gene detection or sequencing platforms.

In some embodiments of the present invention, a method for counting polynucleotide molecules is provided. The method includes: (a) obtaining a plurality of double-stranded polynucleotide fragments from original polynucleotide molecules; (b) optionally, modifying the ends of the polynucleotide fragments obtained in (a) such that each of the double-stranded polynucleotide fragments has an overhang at the 3′ end and a phosphate group at the 5′ end of each strand; (c) ligating two single-stranded polynucleotide adaptors, or a double-stranded polynucleotide linker, to two ends of each of the double-stranded polynucleotide fragments obtained in (a) or (b), respectively, to form single- or double-stranded circular polynucleotide molecules; (d) optionally, linearly amplifying each of the single- or double-stranded circular polynucleotide molecules to form single-stranded nanoballs; and (e) identifying and quantifying the circular polynucleotide molecules obtained in (c), or the single-stranded nanoballs in (d) if (d) is performed, thereby obtaining a counting of the original polynucleotide molecules. The original polynucleotide molecules can comprise DNA and/or RNA. DNA molecules can be counted using the above outlined procedure. When RNA molecules are to be counted, double-stranded cDNA can be first synthesized using the RNA molecules as templates, followed by the fragmentation of the cDNA and the subsequent procedure.

In some embodiments, the ligation in (c) comprises ligating a first single-stranded polynucleotide adaptor onto one of the two ends of each polynucleotide fragment, and ligating a second single-stranded polynucleotide adaptor to the other of the two ends of each polynucleotide fragment obtained in (a) or (b), to thereby form a single-stranded circular polynucleotide molecule.

In certain embodiments, the first single-stranded polynucleotide adaptor and the second single-stranded polynucleotide adaptor have the same 3′ end overhang, or the first single-stranded polynucleotide adaptor and the second single-stranded polynucleotide adaptor have different 3′ end overhangs. In some embodiments, the first and second single-stranded polynucleotide adaptors each comprise a 3′ chain end on which a topoisomerase enzyme is covalently attached.

In certain embodiments, the amplification in (d) is performed and comprises performing rolling circle amplification using the single-stranded circular polynucleotide molecules obtained in (c) as templates to form single-stranded nanoballs, and performing the rolling circle amplification comprises using a primer complementary to a region of the first or the second single-stranded polynucleotide adaptor.

In certain embodiments, the amplification in (d) is performed and comprises performing rolling circle amplification using the single-stranded circular polynucleotide molecules obtained in (c) as templates, and one or multiple primers complementary to one or multiple regions of a strand of the double-stranded polynucleotide fragments obtained in (a) or (b) to form single-stranded nanoballs.

In some embodiments, the ligation in (c) comprises ligating two ends of one double-stranded polynucleotide linker to two ends of each polynucleotide fragment obtained in (a) or (b), respectively, to form a double-stranded circular polynucleotide molecule. In certain of these embodiments, the two ends of the double-stranded polynucleotide linker have the same 3′ end overhang. In other embodiments, two ends of the double-stranded polynucleotide linker have different 3′ end overhangs.

In certain embodiments, the ligation in (c) comprises using at least a first double-stranded polynucleotide linker and a second double-stranded polynucleotide linker, at least one 3′ end overhang on one strand of the first double-stranded polynucleotide linker is different from at least one 3′ end overhang on one strand of the second double-stranded polynucleotide linker.

In certain embodiments, the ligation in (c) comprises using a double-stranded polynucleotide linker that includes that has a phosphate group at the 5′ end of both strands. In other embodiments, the ligation in (c) comprises using a double-stranded polynucleotide linker that includes one strand having a 5′ end with a phosphate group and the other strand having a 5′ end without a phosphate group, whereby the ligation produces a circular double-stranded polynucleotide molecule having a continuous circular strand and a nicked circular strand. In latter embodiments, the amplification in (d) can comprise performing rolling circle amplification using the nicked circular strand as a primer to amplify the continuous circular strand as a template to form single-stranded nanoballs.

In certain embodiments, the amplification in (d) is performed and comprises performing rolling circle amplification using at least one strand of the double-stranded circular polynucleotide molecules obtained in (c) as templates to form single-stranded nanoballs, and performing the rolling circle amplification comprises using a primer complementary to a region of a strand of the double-stranded polynucleotide linker.

In certain embodiments, the amplification in (d) is performed and comprises performing rolling circle amplification using at least one strand of the double-stranded circular polynucleotide molecules obtained in (c) as templates to form single-stranded nanoballs, and performing the rolling circle amplification comprises using one or multiple primers complementary to one or multiple regions of a strand of the double-stranded polynucleotide fragments obtained in (a) or (b).

In some embodiments, the method further comprises: prior to (d), preferentially digesting circular polynucleotide molecules containing methylated nucleotides, if present, in the double-stranded circular polynucleotide molecules, to thereby produce non-circular polynucleotide segments, and then removing the non-circular polynucleotide segments.

The identification and quantification in (e) can comprises hybridizing the circular polynucleotide molecules obtained in (c), or hybridizing the single-stranded molecules obtained in (d) if (d) is performed, to microarrays. The hybridization can also be done against fluorescence dye-conjugated molecular beacons or scorpions. Alternatively, the identification and quantification in (e) comprises sequencing the circular polynucleotide molecules obtained in (c), or sequencing the single-stranded nanoballs obtained in (d) if (d) is performed.

Other features and advantages will become apparent after reviewing the detailed description of the embodiments set forth below.

### BRIEF DESCRIPTION OF THE DRAWINGS

Embodiments will now be described by way of examples, with reference to the accompanying drawings which are meant to be exemplary and not limiting. For all figures mentioned herein, like numbered elements refer to like elements throughout.

FIG. 1 depicts an overview of a process for counting of polynucleotide molecules in accordance with some embodiments of the present invention.

FIGS. 2 and 3 each depict a flowchart for counting of polynucleotide molecules in accordance with some embodiments of the present invention.

FIGS. 4 and 5 depict example processes for counting polynucleotide molecules in accordance with some embodiments of the present invention.

### DETAILED DESCRIPTION

Reference is made in detail to the embodiments herein, examples of which are illustrated in the accompanying drawings. While the invention is described in conjunction with the embodiments below, it is understood that the embodiments described herein are not intended to limit the invention, but are intended to cover alternatives, modifications and equivalents, which are encompassed within the spirit and scope of the invention as defined by the appended claims.

Furthermore, in the following detailed description of the present invention, numerous specific details are set forth in order to more fully illustrate the present invention. It is apparent to one of ordinary skill in the prior art having the benefit of this disclosure that the present invention can be practiced without these specific details. In other instances, well-known methods and procedures, components and processes have not been described in detail so as not to unnecessarily obscure aspects of the present invention. It is appreciated that in the development of any such actual implementation, numerous implementation-specific decisions must be made to achieve the developer's specific goals, such as compliance with application and business related constraints, and that these specific goals vary from one implementation to another and from one developer to another. Moreover, it is appreciated that such a development effort can be complex and time-consuming, but is nevertheless a routine undertaking of engineering for those of ordinary skill in the art having the benefit of this disclosure.

In general, embodiments of the present invention provide methods to manipulate polynucleotides for obtaining single-molecule counting of the number of the polynucleotides. The disclosed methods are simple and low cost, have high sensitivity, specificity, and precision, allow a very small amount of input samples to be tested, and offer short sample to data turnaround time, and is therefore applicable to single gene detection or targeted multiplexing or genome-wide applications.

As used herein, the term “nucleotide” refers to a subunit of a nucleic acid. The term “polynucleotide” includes a single-stranded (ss) or double-stranded (ds) DNA and RNA. For example, cDNA is a type of DNA synthesized from an ss-RNA. A “nucleotide analogue” as used herein is in general a compound in which one or more of the three moieties of a nucleotide (a phosphate backbone, a pentose sugar, either ribose or deoxyribose, and one of four nucleobases) is modified, for example, by attachment of one or more sub stituents and/or by replacement of one or more of the skeletal atoms. A nucleotide analogue functions in a manner similar or analogous to a naturally occurring nucleotide.

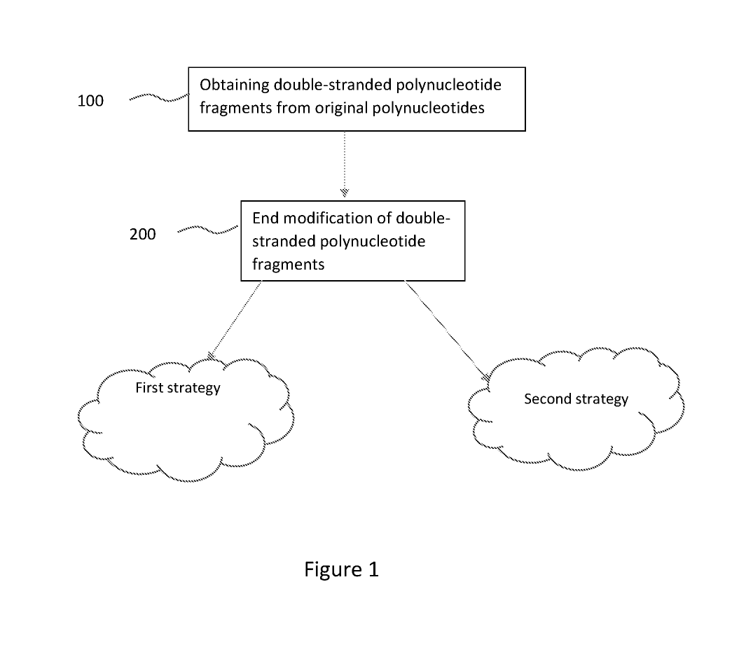

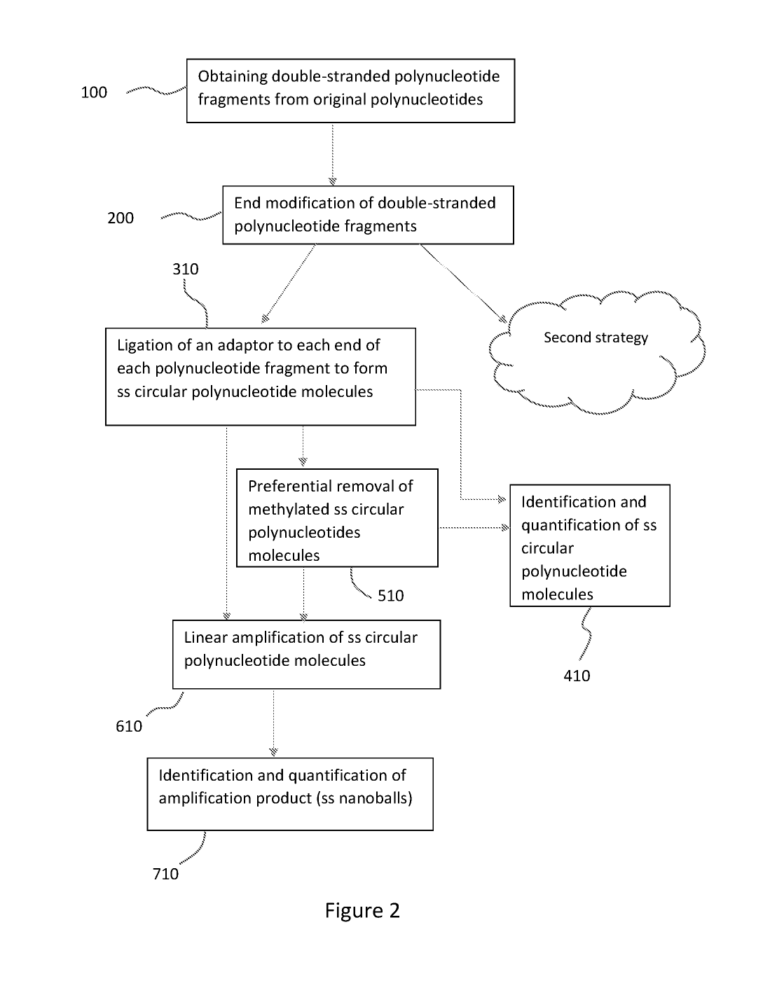

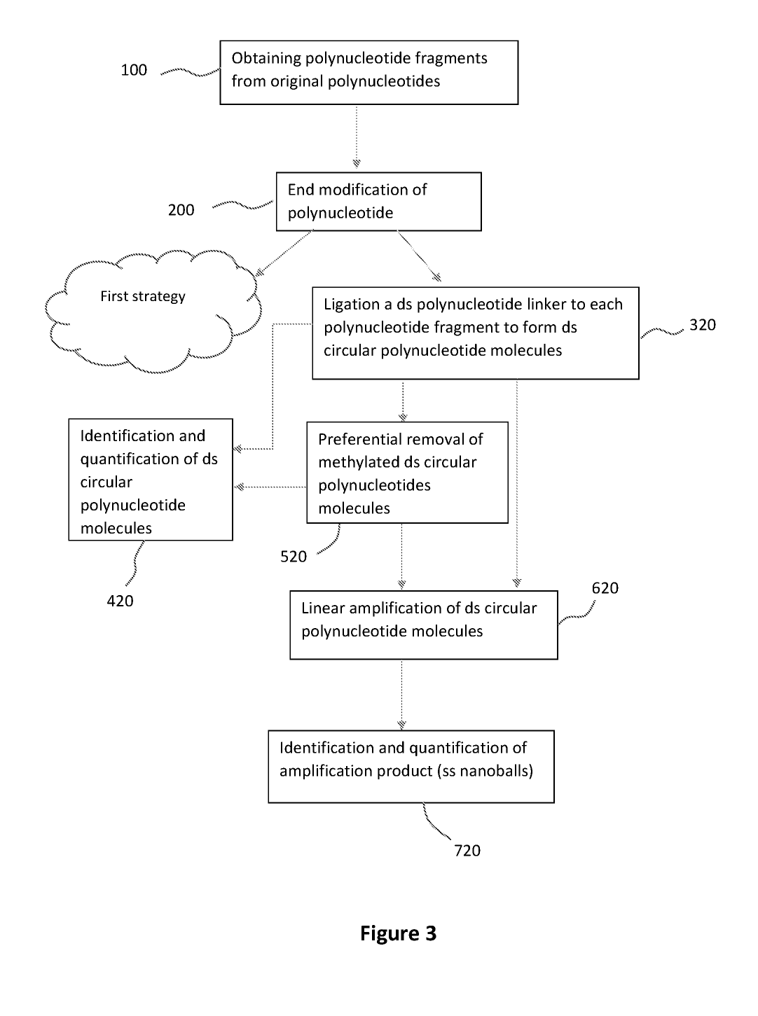

FIGS. 1-3 illustrate an overview of a general process for counting single polynucleotide molecules according to some embodiments of the present invention. First, at step 100, a plurality of double-stranded polynucleotide fragments are obtained from original single- or double-stranded polynucleotide molecules. At step 200, the ends of the double-stranded polynucleotide fragments can be modified or repaired to incorporate phosphate groups and appropriate overhangs which improve efficiency for the subsequent ligation. Thereafter, the process branches into two alternative strategies (a first strategy and a second strategy) based on how the polynucleotide fragments are made into circular polynucleotides. The details of the steps according to the first strategy and the second strategy are illustrated in FIG. 2 and FIG. 3, respectively.

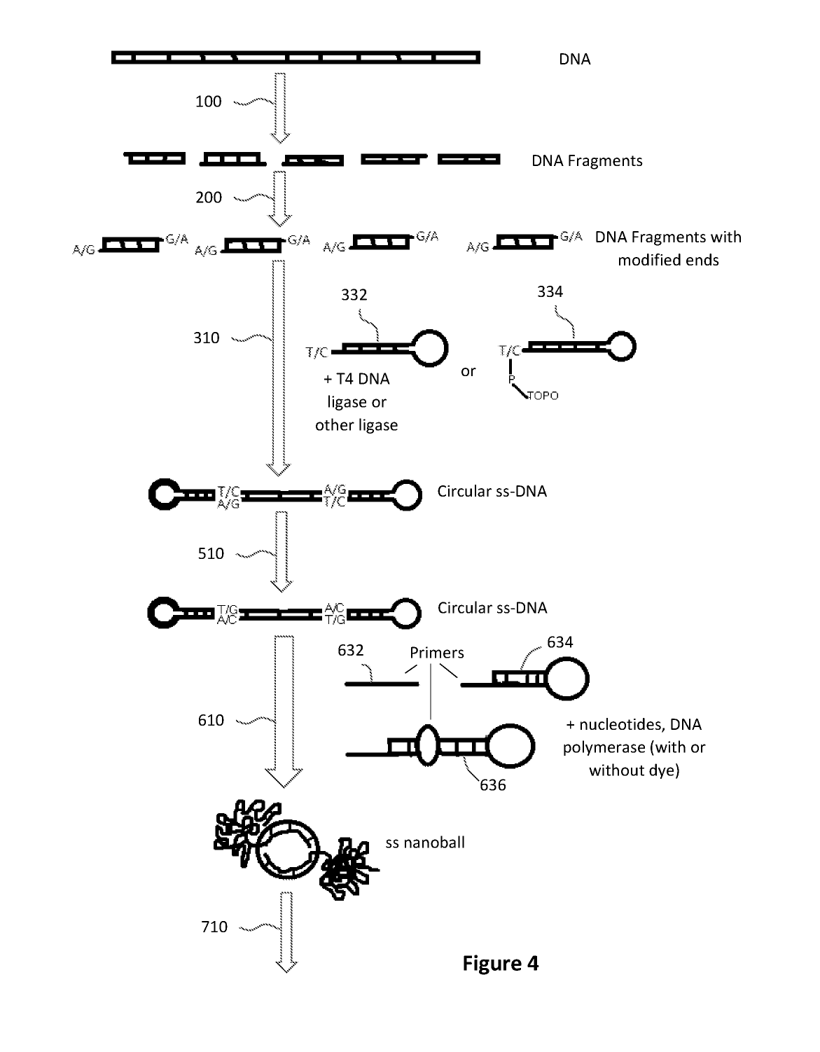

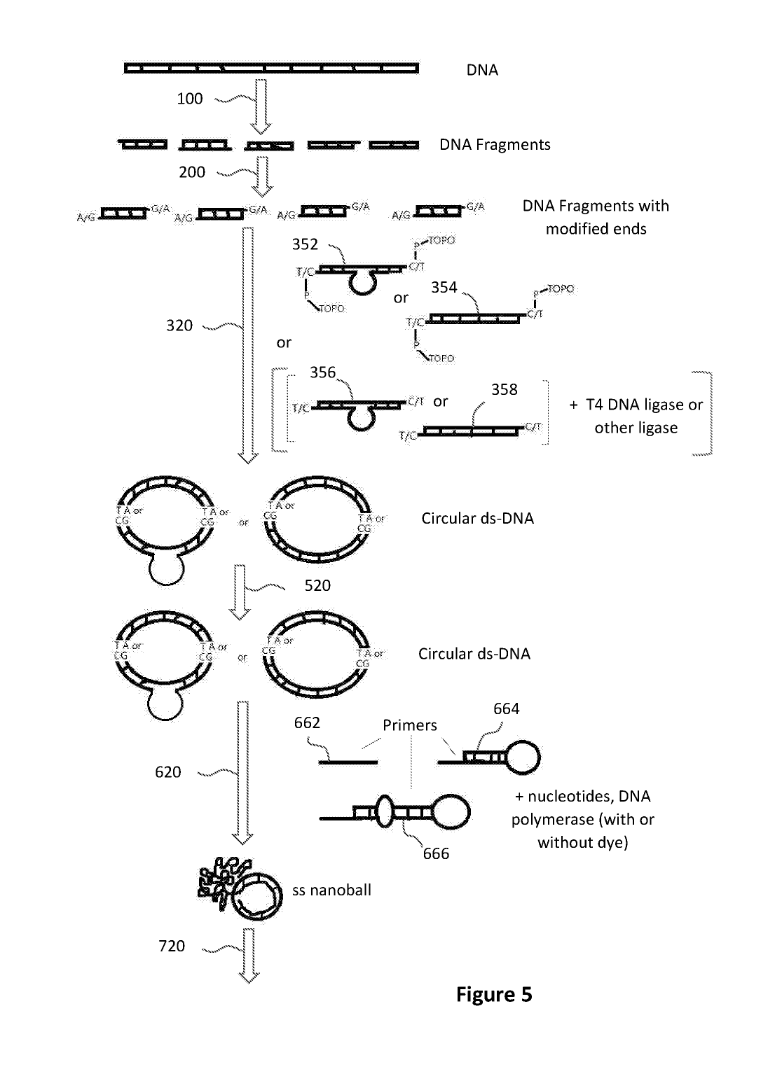

FIGS. 4 and 5 depict example processes according to the first strategy and second strategy, respectively. As discussed above in connection with FIG. 1, it is understood that not all steps illustrated in FIGS. 4 and 5 are needed, and different reagents may be used and different steps may be taken. Hereinbelow, detailed description of the processes and variations in the reagents used in the process as well as the steps performed in the processes are provided in conjunction with FIGS. 1-5.

In the first strategy, and referring to FIG. 2 and FIG. 4, at step 310, the two ends of each double-stranded polynucleotide fragment obtained at step 100 or 200 are ligated to two identical or different single-stranded polynucleotide adaptors each containing a 5′ phosphate, thereby forming a single-stranded circular polynucleotide molecule. At step 410, the single-stranded circular polynucleotide molecules obtained from step 310 are identified and quantified on any suitable gene detection platform, from which the counting of the original double-stranded polynucleotide molecules can be obtained. Alternatively, at step 610, the single-stranded circular polynucleotide molecules are linearly amplified to form single-stranded polynucleotide nanoballs. By linear amplification, it is meant the amplicon grows along the replication template in a linear fashion, rather than the template being replicated in multiple cycles of repeated heating and cooling which results in an exponential growth of the number of copies of the templates. The thus obtained amplification products, the single-stranded nanoballs, are then subject to a suitable gene detection platform for identification and quantification (at step 710), from which the counting of the original double-stranded polynucleotide molecules can be obtained. Optionally, before performing the amplification at step 610, methylated circular polynucleotides can be preferentially removed at step 510.

In the second strategy, and referring to FIG. 3 and FIG. 5, at step 320, two ends of a double-stranded polynucleotide linker (each containing a 5′ phosphate on each of the two strands as exemplified in Example 3) are ligated to the two ends of a single double-stranded polynucleotide fragment (obtained at step 100 or 200), respectively, to form a double-stranded circular polynucleotide molecule. At step 420, the double-stranded circular polynucleotide molecules obtained from step 320 are identified and quantified on any suitable gene detection platform, from which the counting of the original double-stranded polynucleotide molecules can be obtained. Optionally, at step 620, the double-stranded circular polynucleotide molecules are linearly amplified to form single-stranded polynucleotide nanoballs using one of the strands as amplification template, and a primer or multiple primers complementary to any region of the circular polynucleotide molecules. The thus obtained amplification products, the single-stranded nanoballs, are then subject to a suitable gene detection platform for identification and quantification (at step 720), from which the counting of the original double-stranded polynucleotide molecules can be obtained. Further optionally, before performing the amplification at step 620, methylated circular polynucleotides can be preferentially removed at step 520.

Alternatively, in the second strategy, and referring to FIG. 3 and FIG. 5, at step 320, two ends of a double-stranded polynucleotide linker where only one of the two strands contains a 5′ phosphate, as exemplified in Example 2, are ligated to link the two ends of one strand of the double-stranded polynucleotide fragment (obtained at step 100 or 200), and ligated to only one end of the other strand, to form a double-stranded circular polynucleotide molecule with one circular strand having complete continuity while the other strand contains a unlinked nick at the location where the 5′ phosphate group is missing. At step 420, either the continuously circular strand or the nicked strand of the circular polynucleotide molecules obtained from step 320 are identified and quantified on any suitable gene detection platform, from which the counting of the original double-stranded polynucleotide molecules can be obtained. Optionally, at step 620, the continuously circular strand of the circular polynucleotide molecules is linearly amplified to form single-stranded polynucleotide nanoballs using the continuously circular strand (circle) as amplification template, and the other strand that has the nick as the primer. The thus obtained amplification products, the single-stranded nanoballs, are then subject to a suitable gene detection platform for identification and quantification (at step 720), from which the counting of the original double-stranded polynucleotide molecules can be obtained. Further optionally, before performing the amplification at step 620, methylated circular polynucleotides can be preferentially removed at step 520.

Obtaining double-stranded polynucleotide fragments from original polynucleotide molecules at step 100 can be performed by cell lysis, DNA/RNA purification from cells or any tissues; for counting RNA, double-stranded cDNA synthesis based on the RNA as templates; DNA/cDNA fragmentation using physical disruption, enzyme digestion or chemical treatment which includes but is not limited to bioruptors, heating, CviRI, MaeII, AluI, DNase I or any other enzymes, fractionation/isolation from any organism (e.g. human), or any sample originated or derived from an organism (e.g. an exosome, blood plasma, and a body fluid such as urine), or cell lysis or cell culture medium using approaches including but not limited to gel electrophoresis, and capillary electrophoresis. The obtained fragments may have blunted ends, or have an overhang of various nucleotides at the 3′ end and a phosphate group at the 5′ end of each strand.

Modifying the ends (or end repairs) of the polynucleotide fragments at step 200 may include addition of a phosphate at the 5′ ends of both strands by using a kinase, and blunt ending the fragments with enzymes such as T4 DNA polymerase. For example, terminal unpaired nucleotides may be removed from DNA ends by using an enzyme with exonuclease activity, which hydrolyzes a terminal phosphodiester bond, thereby removing the overhang one base at a time. DNA fragments with 5′ overhangs may be blunted by filling in a recessed 3′ terminus with DNA polymerase in the presence of dNTPs. End removal or fill-in can be accomplished using a number of enzymes, including DNA Polymerase I Large (Klenow) Fragment (NEB #M0210), T4 DNA Polymerase (NEB #M0203) or Mung Bean Nuclease (NEB #M0250). Overhangs having one or more nucleotide units created by restriction digestion may directly be used in the ligation. Alternatively, overhang(s) can be added to the 3′ ends of double-stranded DNA/cDNA fragments by KlenTaq/Taq DNA polymerase or any other enzymatic or chemical reaction (optional when TOPO ligation is used). As a result, each fragment may have the same or different overhangs on either end. For example, the end repair techniques of the polynucleotide fragments often result in polynucleotide fragments having a distribution of overhangs on either end (e.g., a fragment may have a dA overhang on the 3′ end of both strands, a dA overhang on 3′ end of one strand and a dG overhang on 3′ end of the other strand, or a dG overhang on the 3′ end of both strands).

In some embodiments, the end repair described above is not performed, and the polynucleotide fragments obtained from the fragmentation of the original polynucleotides are directly ligated by single-stranded polynucleotide adaptors (as exemplified by 332 and 334 in FIG. 4, self-annealed with or without loop structures) or double-stranded polynucleotide linkers which are further explained below.

As illustrated in FIG. 4, at step 310, both ends of each double-stranded polynucleotide fragment are ligated to a respective polynucleotide adaptor with the aid of T4 DNA ligase, topoisomerase (e.g. Vaccinia topoisomerase I), ampligase, E. coli ligase, or other suitable chemical linkage method to form single-stranded circular polynucleotides (also referred to as single-stranded dumbbells or nanorings). An adaptor is a single-stranded polynucleotide having a turn and with two regions (usually complementary in nucleotide sequence when read in opposite directions) on opposing side of the turn capable of forming base-pairs. In some cases, the turn may take the form of a loop structure consisting of unpaired nucleotides. In other cases, an adaptor does not have a loop structure. An adaptor can include a barcode sequence for sample indexing and multiplexing of different samples, and/or include a sequence complimentary to a polynucleotide suitable for rolling circle amplification and sequencing. The 3′ end region of the adaptor may have an overhang composed of one or more nucleotides of the same or different (e.g., single or multiple dT or dC) or their analogues complementary to the overhangs of the double-stranded polynucleotide fragments. The two adaptors ligated on either end of one polynucleotide fragment can be the same or different. For example, the two adaptors can have identical sequences throughout, including the end overhangs, or different sequences elsewhere except the same end overhangs, or the same sequences elsewhere but different overhangs. For ligation of the fragments that have a distribution of overhangs on either end (as discussed above) to form nanorings, a single adaptor or a mixture of adaptors that have different overhangs complementary to the overhangs to those of the polynucleotide fragments may be used. As an example, the adaptors used in the ligation can be a mixture of both adaptors having a dT overhang on the 3′ end and adaptors having a dC overhang on the 3′ end. Further, the 5′ end of each adaptor can have a phosphate, and the components of each adaptor can be any type of nucleotides or their analogues. The bonds connecting the components of each adaptor can be phosphodiester bonds or any other type of bonds. The adaptors can each include a 3′ chain end overhang (such as dT or dC) on which a topoisomerase enzyme is covalently attached (see 334 in FIG. 4). In this case, the ligation reaction may not need additional T4 NDA ligase or another ligase.

As illustrated in FIGS. 2 and 4, at step 410, the single-stranded nanorings are processed by a suitable gene detection platform for identification and quantification, e.g., by hybridization to microarrays or by sequencing. For example, the single-stranded nanorings can be hybridized to microarrays, the microarrays are washed, and scanned. Or the single-stranded nanorings can be hybridized to fluorescence dye-conjugated molecular beacons or scorpions. Or the single-stranded nanorings can be processed by a molecular counting platform including but not limited to next generation sequencing, real time PCR, and bead reader.

As illustrated in FIGS. 2 and 4, at step 610, the amplification can be a rolling circle amplification in the presence or absence of one or more fluorescent DNA binding dyes, with one or more than one primer, such as a universal labeled/unlabelled primer/probe or multiplex (more than one) primers/probes with secondary structures (634, 636) or without secondary structures (632), nucleotides and/or their analogues (such as dNTPs, ribonucleotides, locked nucleic acid (LNA), biotinylated dNTPs, dNTPs conjugated with various reporter dyes), and Phi29 or any other DNA polymerases (e.g., Bst), to form single-stranded DNA nanoballs. Each of such single-stranded DNA nanoball is composed of tandem repeats of {(adaptor)-(positive strand of the original double-stranded fragment)-(adaptor)-(negative strand of the original double-stranded fragment)}, which provides a much stronger signal in hybridization than a single non-repeat sequence in platforms such as microarrays, or error correction in platforms such as next generation sequencing.

It is noted that shown in FIG. 4 the example amplification product contains two “balls” from one template. This result is produced by using a primer sequence complimentary to a region of the adaptor, and the polynucleotide fragments are ligated to two identical adaptors on either end, wherein the primers attach to both adaptors to initiate amplification at two different locations. However, if the two ends are ligated to different adaptors and a primer complimentary to only one of the adaptors is used in amplification, only one ball is produced on each nanoring template.

In some embodiments, the amplification of step 610 can use a single primer complementary to a single region of a strand of a particular double-stranded polynucleotide fragment. In some embodiments, the amplification of step 610 can use a mixture of multiple primers each complementary to a single region of a strand of a particular double-stranded polynucleotide fragment to be analyzed/counted.

As illustrated in FIGS. 2 and 4, at step 710, the amplification products, the single-stranded nanoballs, are processed by a suitable gene detection platform for identification and quantification, e.g., by hybridization to microarrays or by sequencing. For example, the nanoballs can be hybridized to microarrays, the microarrays are washed, and scanned. Or the nanoballs can be fed into a molecular counting platform including but not limited to next generation sequencing, real time PCR, and bead reader.

As illustrated in FIGS. 3 and 5, at step 320, the two ends of a double-stranded DNA linker are respectively connected to the two ends of a double-stranded DNA fragment with T4 DNA ligase, topoisomerase (e.g. Vaccinia topoisomerase I), ampligase, E. coli ligase, or other suitable chemical linkage method to form double-stranded circular polynucleotides (also referred to as double-stranded nanorings). The polynucleotide linker can include a 3′ chain end overhang (such as dT or dC) on each strand on which a topoisomerase enzyme is covalently attached (see 352, 354), in which case the ligation reaction may not need additional T4 NDA ligase or another ligase. The 3′ overhangs of the linker can be single or multiple nucleotides and/or nucleotide analogues of any type that are complementary to the 3′ overhangs of the double-stranded polynucleotide fragments to be counted. For example, the double-stranded DNA linker can have single dT or dC overhang at the 3′ end of each strand, e.g., it can have a single dT overhang at the 3′ end of each strand, a single dC overhang at the 3′ end of each strand, or a single dC overhang at the 3′ end of one strand and a single dT overhang at the 3′ end of the other strand. For ligation, any of these double-stranded DNA linkers can be used alone or their mixtures can be used.

Further, a double-stranded DNA linker can have a phosphate group at the 5′ end of each strand, or a phosphate group at the 5′ end of only one of the two strands. As the 5′ end phosphate group is needed for the ligation, in the latter case, the ligation product will be a double stranded circular polynucleotide molecule or nanoring with a nick at the ligation location where there is no phosphate group on the double-stranded DNA linker. In other words, the nanoring thus formed will have one strand that is completely continuously circular and the other strand having a nick (or discontinuity) at the ligation site where a 5′ phosphate group is missing. The phosphate group can be introduced during oligonucleotide synthesis or any other approaches that are not specified here.

The double-stranded DNA linker can include two polynucleotide strands that are perfectly matched except the end overhangs (354 or 358), or two polynucleotide strands that include at least one secondary structure formed by a plurality of mismatched nucleotide residues on at least one strand (352 or 356). The two ends of the polynucleotide linker can include an overhang of a single or multiple dC or dT on the 3′ end on each strand (352, 354, 356, 358). The components of each linker can be any type of nucleotides or their analogues. The bonds connecting the components of each strand of the linker can be phosphodiester bonds or any other type of bonds. Further, the two ends of the polynucleotide linker can be each covalently attached with a topoisomerase enzyme (352 or 354). In this case, the ligation reaction may not need additional T4 NDA ligase or another ligase. Further, the polynucleotide linker can include a barcode sequence for sample indexing multiplexing of different samples, and/or include a sequence on one of its strands that is complimentary to a polynucleotide suitable for rolling circle amplification and sequencing.

As illustrated in FIGS. 3 and 5, at step 420, the double-stranded nanorings are processed by a suitable gene detection platform for identification and quantification, e.g., by hybridization to microarrays or by sequencing. For example, the double-stranded nanorings can be hybridized to microarrays, the microarrays are washed, and scanned. Or the double-stranded nanorings can be hybridized to fluorescence dye-conjugated molecular beacons or scorpions. Or the double-stranded nanorings can be processed by a molecular counting platform, which includes but is not limited to next generation sequencing, real time PCR, and bead reader.

As illustrated in FIGS. 3 and 5, at step 620, the amplification can be a rolling circle amplification in the presence or absence of one or more fluorescent DNA binding dyes, with one or more than one primer, such as a universal labeled/unlabelled primer or multiplex (more than one) primers with secondary structures (664, 666) or without secondary structures (662), nucleotides and/or analogues thereof (such as dNTPs, ribonucleotides, locked nucleic acid (LNA), biotinylated dNTPs, dNTPs conjugated with various reporter dyes) and Phi29 or any other DNA polymerases (e.g., Bst) to form single-stranded DNA nanoballs. In some embodiments, this amplification can use one or multiple primers complementary to one or multiple regions of a strand of the double-stranded polynucleotide linker. In other embodiments, this amplification can use one or multiple primers complementary to one or multiple regions of a strand of the double-stranded polynucleotide fragments to be analyzed/counted. In some embodiments, when the double-stranded nanoring has a nicked strand as described above, the amplification can use the nicked strand as a primer, and the other strand as the template for amplification.

As illustrated in FIGS. 3 and 5, at step 720, the amplification products, the single-stranded nanoballs, are processed by a suitable gene detection platform for identification and quantification, e.g., by hybridization to microarrays or by sequencing. For example, the nanoballs can be hybridized to microarrays, the microarrays are washed, and scanned. Or the nanoballs can be fed into a molecular counting platform including but not limited to next generation sequencing, real time PCR, and bead reader.

Before subjecting the nanorings for identification/quantification on any gene detection platform (at step 410 or 420) or performing rolling circle amplification (at step 610 or 620), circular polynucleotide molecules with higher degree of methylation can be preferentially removed, e.g., by using methylation-sensitive enzyme(s) to break down the circular polynucleotide molecules, and remove the resulting non-circular polynucleotide segments, e.g., by using exonuclease(s). It is noted that before denaturing, the single-stranded circular polynucleotide molecules (the dumbbell-shaped nanorings illustrated in FIG. 4) still retain a double-stranded configuration except at the two loop ends, and are therefore susceptible to the action of methylation-sensitive enzyme(s). This step can be particularly important for gene analysis for a fetus by using circulating DNAs in the blood of the pregnant mother bearing the fetus, because the mother's DNA has higher degree/frequency of methylation, and therefore removal of at least portions of the mother's DNA from the sample can improve the efficiency of the detection.

In the amplification of either step 610 or step 620, the primer sequence(s) for the rolling circle amplification can have any suitable adaptor sequence(s) at the 5′ end of the primer for sample indexing and molecule counting (e.g. sequencing) on platform including but not limited to next generation sequencer. The primers can be composed of units including but not limited to deoxyribonucleotide, ribonucleotide, Locked Nucleic Acid (LNA), and any of their modified analogues. The DNA-binding reporter dyes include but not limited to SYBR Green, Propidium iodide, YO-PRO-1, and TOTO-3, or molecular beacon or scorpion probes with different reporter dye and quencher combination for DNA identification and counting on platforms including but not limited to microarrays, flow cytometers, bead counters, and droplet readers. Incorporation of biotin-conjugated dNTP into the nanoballs can allow the nanoballs to be visualized with reagents such as streptavidin-conjugated antibodies and SAPE.

For example, at either step 410 or 420, the single-stranded or double-stranded nanorings may be sequenced in a rolling circle manner for sequencing error correction on single molecule sequencing platforms from companies including but not limited to Pacific Biosciences, or a nanopore-based platform offered by Genia, where a protein pore embedded in a lipid bilayer membrane constitute the nanopore.

For example, at either step 710 or 720, the nanoballs can be sequenced on the platforms including but not limited to MinION from Oxford Nanopore, and the instruments from other companies including but not limited to Pacific Biosciences and BGI, where one nanoball can be completely sequenced in a nanopore or a nanoring can be sequenced repeatedly in a rolling circle manner for sequencing error correction. This is because the existence of the tandem repeat sequences of fragments in the nanoball allows multiple scan of the same sequence, thereby correcting reading errors by correlating the results from the multiple scans.

The following non-limiting Examples are provided to further illustrate certain aspects of the present invention.

### EXAMPLE 1

### Creation of a Genome-Wide Single-Stranded DNA Nanoring Library by Ligating Mixtures of Single-Stranded Adaptors onto both Ends of each Genomic DNA Fragment of the Genome

In a PCR tube, add 10 ng end-repaired human genomic DNA fragments (200-500 bp in length) each containing a 5′ phosphate and a single 3′ dA or dG overhang, 2 μl T4 DNA Ligase Buffer (10×), 10 ng single-stranded adaptor oligonucleotide 1 (SEQ ID No. 1) and 10 ng single-stranded adaptor oligonucleotide 2 (SEQ ID No. 2) and 1 μl T4 DNA Ligase. Bring the total volume to 20 μl with Nuclease-free water. Incubate the reaction at room temperature for 10 minutes. Heat inactivate at 65° C. for 10 minutes.

```
SEQ ID No. 1:
Single-stranded adaptor oligonucleotide 1
  ACTCTTTCCCTACACGACGCTCTTCCGATCTTCGCATTCCGATCT3′
C |||||||||||||||||||
  ATCTAGAGCCACCAGCGGCATAGTAGCTAGAAGCGTAAGGCTAG-P5′
SEQ ID No. 2:
Single-stranded adaptor oligonucleotide 2
  ACTCTTTCCCTACACGACGCTCTTCCGATCTTCGCATTCCGATCC3′
C |||||||||||||||||||
  ATCTAGAGCCACCAGCGGCATAGTAGCTAGAAGCGTAAGGCTAG-P5′
```

### EXAMPLE 2

### Creation of a Genome-Wide Double-Stranded DNA Nanoring Library (with a Nick on One of the Two Strands of each Nanoring) by Ligating Mixtures of Double-Stranded Polynucleotide Linkers having a Hairpin Secondary Structure to Connect both Ends of One Strand and One End of the Other Strand of Each Genomic DNA Fragment

In a PCR tube, add 10 ng end-repaired human genomic DNA fragments (200-500 bp in length) each containing a 5′ phosphate and a single 3′ dA or dG overhang, 2 μl T4 DNA Ligase Buffer (10×), 10 ng double-stranded polynucleotide linker L1 (composed of SEQ ID Nos. 3 and 4 as shown below) and 10 ng double-stranded polynucleotide linker L2 (composed of SEQ ID Nos. 5 and 6 as shown below) and 1 μl T4 DNA Ligase. Bring the total volume to 20 μl with Nuclease-free water. Incubate the reaction at room temperature for 10 minutes. Heat inactivate at 65° C. for 10 minutes.

Double-Stranded Polynucleotide Linker (L1) having a Hairpin Secondary Structure in One Strand

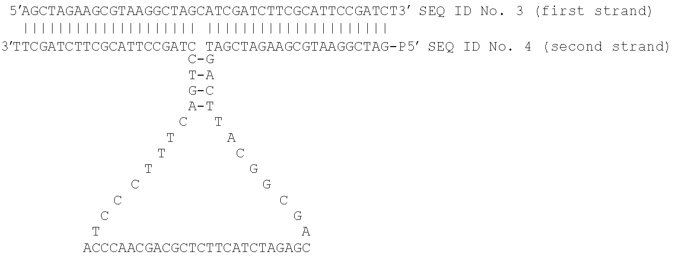

Double-Stranded Polynucleotide Linker (L2) having a Hairpin Secondary Structure in One Strand

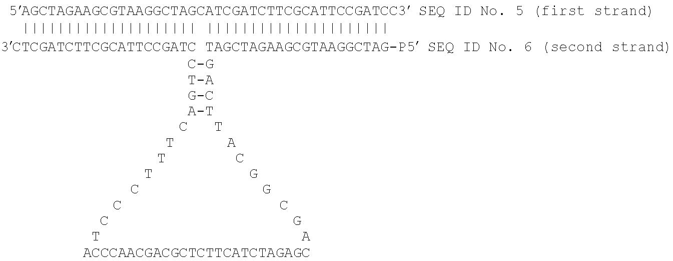

Both linker L1 and linker L2 have two double-stranded arms formed by paired nucleotides, and a hairpin secondary structure formed by a segment of polynucleotides in the middle of the lower strand. Linker L1 has a single dT overhang at the 3′ end of each strand, and linker L2 has a single dC overhang at the 3′ end of each strand.

Also, linker L1 and linker L2 each have a phosphate group on a 5′ end of its lower strand, and the 5′ end of its upper strand does not have a phosphate group. Such a structure results in the ligation product (circular double-stranded polynucleotide or nanoring) having one strand with complete continuity throughout the circle or nanoring and the other strand having a discontinuity or nick at the position of the ligation where the phosphate group is missing.

### EXAMPLE 3

### Creation of a Genome-Wide Double-Stranded DNA Nanoring Library by Ligating Mixtures of Double-Stranded Adaptors to Connect both Ends of Each Genomic DNA Fragment

In a PCR tube, add 10 ng end-repaired human genomic DNA fragments (200-500 bp in length) each containing a 5′ phosphate and a single 3′ dA or dG overhang, 2 μl T4 DNA Ligase Buffer (10×), 10 ng double-stranded polynucleotide linker having a hairpin secondary structure, L3 (composed of SEQ ID Nos. 7 and 8) and 10 ng Double-stranded polynucleotide linker having a hairpin secondary structure, L4 (composed of SEQ ID Nos. 9 and 10) and 1 μl T4 DNA Ligase. Bring the total volume to 20 μl with Nuclease-free water. Incubate the reaction at room temperature for 10 minutes. Heat inactivate at 65° C. for 10 minutes.

Double-Stranded Polynucleotide Linker L3 (with a Hairpin Secondary Structure in One Strand)

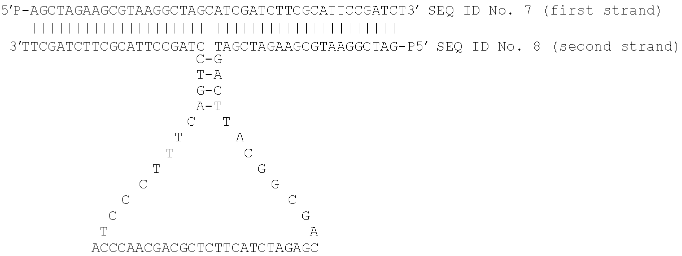

Double-Stranded Polynucleotide Linker L4 (with a Hairpin Secondary Structure in One Strand)

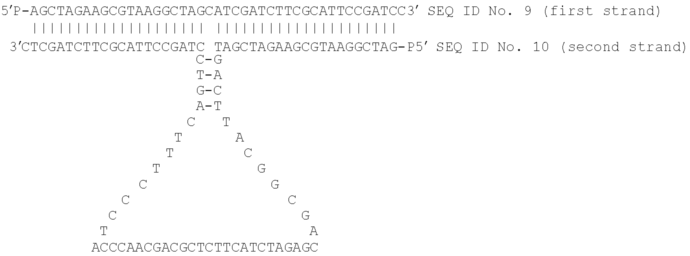

It is noted that both linker L3 and linker L4 have two double-stranded arms formed by paired nucleotides, and a hairpin secondary structure formed by a segment of polynucleotides in the middle of the lower strand. Linker L3 has a single dT overhang at the 3′ end of each strand, and linker L4 has a single dC overhang at the 3′ end of each strand. Also, linker L3 and linker L4 each have a phosphate group on a 5′ end of each of the strands. Thus, as a result of the ligation, the circular double-stranded polynucleotide or nanoring formed will have complete continuity on each strand throughout the circle or nanoring.

### EXAMPLE 4

### Creation of a Genome-Wide Single-Stranded DNA Nanoball Library by Rolling Circle Amplification using the Genome-Wide Single-stranded Nanoring Library Generated in Example 1 and a Polynucleotide Primer

In a PCR tube, mix the genome-wide single-stranded nanoring library generated in Example 1 with 0.5 μl primer (400 ng/μl, SEQ ID No. 11), 0.3 μl Phi29 DNA polymerase (10 U/μl, New England Biolabs), 2 μl of 10× Phi29 DNA polymerase buffer, 0.2 μl of 100× BSA, 3.2 μl of 2.5 mM dNTP and 1 μl of 20% DMSO (Sigma Aldrich) in a volume of 20 μl. Incubate at 30° C. for 24 h, and then at 65° C. for 10 min.

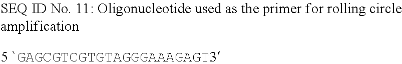

### EXAMPLE 5

### Creation of a Genome-Wide Single-Stranded DNA Nanoball Library by Rolling Circle Amplification using the Genome-Wide Single-Stranded Nanoring Library Generated in Example 2 and Nicked Complementary Strand of the Nanorings as Primers

In a PCR tube, mix the genome-wide single-stranded nanoring library generated in Example 2 with 0.3 μl Phi29 DNA polymerase (10 U/μl, New England Biolabs), 2 μl of 10× Phi29 DNA polymerase buffer, 0.2 μl of 100× BSA, 3.2 μl of 2.5 mM dNTP and 1 μl of 20% DMSO (Sigma Aldrich) in a volume of 20 μl Incubate at 30° C. for 24 h, and then at 65° C. for 10 min.

As described above, the nanorings in the nanoring library in Example 2 include a nicked structure in the circular double-stranded DNA nanoring. The rolling circle amplification can use the strand that does not have the nick as a template for amplification. The nicked strand can be used as a primer for such amplification.

### EXAMPLE 6

### Creation of a Genome-Wide Single-Stranded DNA Nanoball Library by Rolling Circle Amplification using the Genome-Wide Double-Stranded Nanoring Library Generated in Example 3 and a Polynucleotide Primer

In a PCR tube, mix the genome-wide double-stranded nanoring library generated in Example 3 with 0.5 μl primer (400 ng/μl, SEQ ID No. 12), 0.3 μl Phi29 DNA polymerase (10 U/μl, New England Biolabs), 2 μl of 10× Phi29 DNA polymerase buffer, 0.2 μl of 100× BSA, 3.2 μl of 2.5 mM dNTP and 1 μl of 20% DMSO (Sigma Aldrich) in a volume of 20 μl. Incubate at 30° C. for 24 h, and then at 65° C. for 10 min.

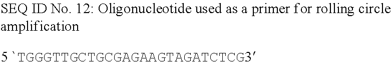

### EXAMPLE 7

### Preferential Removal of Methylated Nanorings from the Genome-Wide Nanoring Libraries Generated in Examples 1 through 3

In a PCR tube, mix the genome-wide nanoring libraries with 3 μl of 10× CutSmart® Buffer, 1 μl 30× Enzyme Activator Solution, 1 μl MspJI (5 unit) in a volume of 30 μl. Incubate at 37° C. for 4 h, and then at 65° C. for 20 min.

The present invention has been described in terms of specific embodiments incorporating details to facilitate the understanding of principles of construction and operation of the invention. Such reference herein to specific embodiments and details thereof is not intended to limit the scope of the claims appended hereto. It is apparent to one skilled in the art that other various modifications can be made in the embodiment chosen for illustration without departing from the spirit and scope of the invention as defined by the claims.

What is claimed is:

1. A method for counting polynucleotide molecules, comprising:
(a) obtaining a plurality of double-stranded polynucleotide fragments from original polynucleotide molecules;
(b) optionally, modifying the ends of the polynucleotide fragments obtained in (a) such that each of the double-stranded polynucleotide fragments has an overhang at the 3′ end and a phosphate group at the 5′ end of each strand;
(c) ligating two single-stranded polynucleotide adaptors, or a double-stranded polynucleotide linker, to two ends of each of the double-stranded polynucleotide fragments obtained in (a) or (b), respectively, to form single- or double-stranded circular polynucleotide molecules;
(d) optionally, linearly amplifying each of the single- or double-stranded circular polynucleotide molecules to form single-stranded nanoballs; and
(e) identifying and quantifying the circular polynucleotide molecules obtained in (c), or the single-stranded nanoballs in (d) if (d) is performed, thereby obtaining a counting of the original polynucleotide molecules.

2. The method of claim 1, wherein the ligation in (c) comprises ligating a first single-stranded polynucleotide adaptor onto one of the two ends of each polynucleotide fragment, and ligating a second single-stranded polynucleotide adaptor to the other of the two ends of each polynucleotide fragment obtained in (a) or (b), to thereby form a single-stranded circular polynucleotide molecule.

3. The method of claim 2, wherein the first single-stranded polynucleotide adaptor and the second single-stranded polynucleotide adaptor has a same 3′ end overhang, or the first single-stranded polynucleotide adaptor and the second single-stranded polynucleotide adaptor has different 3′ end overhangs.

4. The method of claim 2, wherein the first and second single-stranded polynucleotide adaptors each comprise a 3′ chain end on which a topoisomerase enzyme is covalently attached.

5. The method of claim 2, wherein the amplification in (d) is performed and comprises performing rolling circle amplification using the single-stranded circular polynucleotide molecules obtained in (c) as templates to form single-stranded nanoballs, and wherein performing the rolling circle amplification comprises using a primer complementary to a region of the first or the second single-stranded polynucleotide adaptor.

6. The method of claim 2, wherein the amplification in (d) is performed and comprises performing rolling circle amplification using the single-stranded circular polynucleotide molecules obtained in (c) as templates to form single-stranded nanoballs, and wherein performing the rolling circle amplification comprises using one or multiple primers complementary to one or multiple regions of a strand of the double-stranded polynucleotide fragments obtained in (a) or (b).

7. The method of claim 1, wherein the ligation in (c) comprises ligating two ends of one double-stranded polynucleotide linker to two ends of each polynucleotide fragment obtained in (a) or (b), respectively, to form a double-stranded circular polynucleotide molecule.

8. The method of claim 7, wherein the two ends of the double-stranded polynucleotide linker have the same 3′ end overhang or different 3′ end overhangs.

9. The method of claim 7, wherein the ligation in (c) comprises using at least a first double-stranded polynucleotide linker and a second double-stranded polynucleotide linker, at least one 3′ end overhang on one strand of the first double-stranded polynucleotide linker is different from at least one 3′ end overhang on one strand of the second double-stranded polynucleotide linker.

10. The method of claim 7, wherein the ligation in (c) comprises using a double-stranded polynucleotide linker that includes one strand having a 5′ end with a phosphate group and the other strand having a 5′ end without a phosphate group, whereby the ligation produces a circular double-stranded polynucleotide molecule having a continuous circular strand and a nicked circular strand.

11. The method of claim 7, wherein the amplification in (d) is performed and comprises performing rolling circle amplification using at least one strand of the double-stranded circular polynucleotide molecules obtained in (c) as a template to form single-stranded nanoballs, and wherein performing the rolling circle amplification comprises using a primer complementary to a region of a strand of the double-stranded polynucleotide linker.

12. The method of claim 7, wherein the amplification in (d) is performed and comprises performing rolling circle amplification using at least one strand of the double-stranded circular polynucleotide molecules obtained in (c) as a template to form single-stranded nanoballs, and wherein performing the rolling circle amplification comprises using one or multiple primers complementary to one or multiple regions of a strand of the double-stranded polynucleotide fragments.

13. The method of claim 10, wherein the amplification in (d) is performed and comprises performing rolling circle amplification using the nicked circular strand as a primer to amplify the continuous circular strand as a template to form single-stranded nanoballs.

14. The method of claim 1, further comprising:
prior to (d), preferentially digesting circular polynucleotide molecules containing methylated nucleotides, if present, in the double-stranded circular polynucleotide molecules, to thereby produce non-circular polynucleotide segments, and removing the non-circular polynucleotide segments.

15. The method of claim 1, wherein the identification and quantification in (e) comprises hybridizing the circular polynucleotide molecules obtained in (c), or hybridizing the single-stranded molecules obtained in (d) if (d) is performed, to microarrays.

16. The method of claim 1, wherein the identification and quantification in (3) comprises hybridizing the circular polynucleotide molecules obtained in (c), or hybridizing the single-stranded nanoballs obtained in (d) if (d) is performed, to fluorescence dye-conjugated molecular beacons or scorpions.

17. The method of claim 1, wherein the identification and quantification in (e) comprises sequencing the circular polynucleotide molecules obtained in (c), or sequencing the single-stranded nanoballs obtained in (d) if (d) is performed.

18. The method of claim 1, wherein the original polynucleotide molecules include DNA molecules.

19. The method of claim 1, wherein the original polynucleotide molecules include RNA molecules.

20. The method of claim 19, wherein obtaining the plurality of double-stranded polynucleotide fragments in (a) comprises:
synthesizing double-stranded cDNA molecules using the RNA molecules as templates; and
fragmenting the double-stranded cDNA molecules to obtain the plurality of double-stranded polynucleotide fragments.
